(19) United States
(12) Patent Application Publication
(10) Pub. No.: US 2014/0100573 A1
(43) Pub. Date: Apr. 10, 2014

(54) APPARATUS AND METHODS FOR SECURING TOGETHER BONE FRAGMENTS

(71) Applicant: NEOS SURGERY, S.L., Cerdanyola Del Valles (ES)

(72) Inventors: Salvador Llas Vargas, Lleida (Lleida) (ES); Lluís Chico Roca, Badalona (Barcelona) (ES); Montserrat Charles-Harris Ferrer, Barcelona (Barcelona) (ES)

(21) Appl. No.: 13/869,748

(22) Filed: Apr. 24, 2013

(30) Foreign Application Priority Data
Oct. 8, 2012 (ES) P201231541

(51) Int. Cl. A61B 17/82

(52) U.S. Cl. CPC A61B 17/823
USPC 606/74

(57) ABSTRACT

An apparatus for securing together first and second bone segments having peripheral surfaces comprising one or more bends. In some implementations the fixation device includes an elongate flexible strap having a proximal end and a distal end, the elongate flexible strap having a plurality of ratchet teeth disposed along an elongate distal portion thereof that function with a ratchet member within a locking head situated at the proximal end to permit the formation of a closed loop about the bone fragments. In some implementations the elongate flexible strap has a first elongate portion situated between the locking head and the elongate distal portion, the first elongate portion of the elongate flexible strap being more flexible than the elongate distal portion of the elongate flexible strap, the first elongate portion configured to extend across at least one of the one or more bends when the apparatus is in a secured state.

### CROSS-REFERENCE TO RELATED APPLICATIONS

This application relates to and claims the benefit and priority to Spanish Patent Application No. P201231541, filed Oct. 8, 2012.

### TECHNICAL FIELD

The disclosure relates to apparatus and methods for securing together bone segments.

### BACKGROUND

Some medical procedures involve the securing together of bone segments. One such procedure occurs in joining the parts of a sternum that have been split in order to gain access to a patient's heart, lungs and the like located in the thoracic cavity of the patient. FIG. 1 illustrates an anterior view of a patient's sternum 20 and associated ribs 28 following a median sternotomy. A cut 22 that runs the length of the sternum 20 typically divides the sternum into first and second halves 24 and 26, respectively. After the surgical procedure is performed, the sternum must be closed so that the two halves 24,26 of the sternum are brought into tight abutment with one another.

Past methods for joining the severed parts of the sternum have included the use of steel wires that are wound around the severed parts and then twisted to place the parts in tight abutment. Other methods involve the use of flexible straps that are looped about the split portions of the sternum and then tightened to affix the split portions together. U.S. Pat. No. 5,462,542 is an example of such a method wherein tightening of the strap is achieved by the surgeon grasping an end of the strap and pulling the strap end in a tensioning direction until the sternum portions are joined together in an adjacent face-to-face contacting relation. A problem with this prior approach is that the ability to tension the straps is limited due to the stiffness of the straps and the small surface area available for being grasped. As a result of the limited ability of the straps to be tensioned arise the problem of there not always being a good conformity of the strap with the bone segments being joined. This can result in the unwanted existence of gaps between the strap and the bone portions which may lead to sternal dehiscence, a condition with serious clinical consequences.

The Sternal ZipFix System™ currently sold by Synthes CMF utilizes a handheld stainless steel application instrument (Part No. 03.501.080) that is slid over the loose end of the flexible strap, and by use of a trigger mechanism operated by the surgeon applies tension to the strap to effectuate a tight joining of the split sternum parts. The flexible strap of the Sternal ZipFix System™, like the flexible strap in the '542 patent above, has a working length (the length of the strap that is intended to be applied against the sternum bone segments) that is straight before being looped around the sternum parts and has a generally uniform flexibility along its length. These characteristics make it difficult for the strap to properly conform to the contour of the sternum. The Synthes® Sternal ZipFix System™ attempts to address this problem with the use of the application instrument that is adapted to apply an ample amount of tension to force the strap into conformity with the parts of the sternum. There are several problems associated with this approach. First, the application instrument must be sterilized after each use which involves time and money resources and a risk of infection to patients if not properly sterilized. Second, in some patients (such as older and female patients) the sternum bone mass may be thin or osteoporotic. In such patients the large amount of force that is required to be applied to the strap in order to achieve conformity with the sternum and to reduce the existence of gaps between the strap and sternum can cause the strap to crush the underlying bone, and may in severe instances result in a cut through the bone.

In certain applications such as, for example, binding two fragments of the sternum, a certain expansion of the bone tissue to be repaired should be provided to accommodate movement such as when the thoracic cage expands on coughing.

### SUMMARY OF THE DISCLOSURE

According to some implementations an apparatus for tightly securing together first and second bone segments having peripheral surfaces is provided that comprises one or more bends, the apparatus comprising: an elongate flexible strap having a proximal end and a distal end and capable of being formed and maintained in a loop configuration about the first and second bone segments; and the elongate flexible strap having a first elongate portion situated between the proximal end and the distal end, the first elongate portion of the elongate flexible strap being more flexible than one or more other portions of the elongate flexible strap, the first elongate portion configured to extend across at least one of the one or more bends of one or both of the first and second bone segments when the apparatus is tightly looped about the first and second bone segments.

According to some implementations an apparatus for tightly securing together first and second bone segments having peripheral surfaces is provided that comprises one or more bends is provided, the apparatus comprising: an elongate flexible strap having a proximal end and a distal end, the elongate flexible strap having a plurality of ratchet teeth disposed along an elongate distal portion thereof; a locking head situated at the proximal end of the elongate flexible strap, the head defining a transverse channel with a ratchet member situated therein, the channel dimensioned to receive the elongate distal portion of the elongate flexible strap, the ratchet member having one or more teeth adapted to engage the ratchet teeth of the elongate flexible strap in a manner that permits movement of the elongate distal portion of the elongate flexible strap through the channel in only one direction, the elongate distal portion of the elongate flexible strap adapted to be advanced through the channel until the apparatus assumes a secured state with the first and second bone segments being tightly secured together; the elongate flexible strap having a first elongate portion situated between the locking head and the elongate distal portion, the first elongate portion of the elongate flexible strap being more flexible than the elongate distal portion of the elongate flexible strap, the first elongate portion configured to extend across at least one of the one or more bends of the first and/or second bone segment when the apparatus is in the secured state.

According to some implementations an apparatus for tightly securing together a first bone segment with a second bone segment is provided, the peripheral surface of each of the first and second bone segments including a top face, a bottom face and a side face interposed between the respective top and bottom faces, the side face of the second bone segment being located opposite the side face of the first bone segment, a first corner existing between the top and side faces of the first bone segment, a second corner existing between the top and side faces of the second bone segment, a third corner existing between the bottom and side faces of the second bone segment, a fourth corner existing between the bottom and side faces of the first bone segment, with the apparatus comprising: an elongate flexible strap having a proximal end and a distal end, the elongate flexible strap having a plurality of ratchet teeth disposed along an elongate distal portion thereof; a locking head situated at the proximal end and unitarily formed as a single piece with the elongate flexible strap, the head defining a transverse channel with a ratchet member situated therein, the channel dimensioned to receive the elongate distal portion of the elongate flexible strap, the ratchet member having one or more teeth adapted to engage the ratchet teeth of the elongate flexible strap in a manner that permits movement of the elongate distal portion of the elongate flexible strap through the channel in only one direction, the elongate distal portion of the elongate flexible strap adapted to be advanced through the channel until the apparatus assumes a secured state with the first and second bone segments being tightly secured together, the locking head being configured to rest on or near the first corner of the first bone segment when the apparatus is in the secured state; the elongate flexible strap having a first elongate portion situated between the locking head and the elongate distal portion, the first elongate portion of the elongate flexible strap being more flexible than the elongate distal portion of the elongate flexible strap, the first elongate portion configured to extend across at least the second and third corners of the second bone segment when the apparatus is in the secured state.

According to some implementations the elongate distal portion of the elongate flexible strap includes features that facilitate the formation of a loop therein suitable for receiving and being gripped by one or more fingers of a human hand.

These and other implementations will be disclosed and made evident in the light of the drawings and the detailed description thereof.

### BRIEF DESCRIPTION OF THE DRAWINGS

FIG. 1 illustrates an anterior view of a typical patient's sternum and associated ribs.

FIG. 2 illustrates a cross-section view of a portion of the sternum along line A-A of FIG. 1.

FIG. 3A illustrates a side view of a fixation device according to an implementation.

FIG. 3B illustrates a top view of the fixation device of FIG. 3A.

FIG. 3C illustrates an isometric view of the fixation device of FIG. 3A.

FIG. 3D illustrates a form of the fixation device of FIG. 1 when partially looped about a sternum bone mass.

FIG. 3E illustrates a fixation device having a locking head with a transverse channel that is arranged substantially parallel to the longitudinal axis of the device.

FIG. 4A illustrates a cross-sectional side view of a head region of a fixation device according to an implementation.

FIG. 4B illustrates a top view of the head region depicted in FIG. 4A.

FIG. 4C illustrates a cross-sectional side view of a locking head according to some implementations.

FIG. 4D illustrates a cross-sectional side view of the locking head depicted in FIG. 3E according to some implementations.

FIG. 5 illustrates a fixation device secured about the bone fragments of a sternum according to an implementation.

FIG. 6A illustrates a side view of a fixation device according to an implementation.

FIG. 6B illustrates a partial perspective view of the fixation devices of FIG. 6A.

FIGS. 7A-7E illustrate exemplary cross-sectional views of the enhanced flexibility region(s) a fixation device according to various implementations.

FIG. 8 illustrates a top view of a fixation device according to an implementation.

FIGS. 9A-B illustrate the dimensional characteristics of the fixation device of FIGS. 6A-B according to some implementations.

FIG. 10 illustrates a fixation device adapted to the formation of a loop at a distal end thereof than can be gripped by a hand of the surgeon according to an implementation.

FIGS. 11A-B illustrate exemplary distal end portions of a fixation device according to some implementations.

FIGS. 12A-B illustrate apparatus and method of applying tension to a strap of a fixation device according to some implementations.

FIG. 13 illustrates apparatus and method of applying tension to a strap of a fixation device according to another implementation.

FIG. 14 illustrates apparatus and method of applying tension to a strap of a fixation device according to another implementation.

FIGS. 15A-B illustrate apparatus and method of applying tension to a strap of a fixation device according to another implementation.

FIG. 16 illustrates an implementation of a bone fragments fixation device having an active region.

FIG. 17 illustrates another implementation of a bone fragments fixation device having an active region.

FIG. 18 illustrates another implementation of a bone fragments fixation device having an active region.

FIGS. 19-22 illustrate different exemplary means of joining an active region to a strap of bone fragments fixation device.

FIG. 23 illustrates a fixation device according to another implementation.

FIG. 24 illustrates a fixation device according to another implementation.

### DETAILED DESCRIPTION

Throughout this disclosure the discussion will be primarily directed to the joining together of sternum bone fragments. It is to be appreciated, however, that the devices and methods disclosed herein are applicable to the joining together of bone fragments other than those of the sternum.

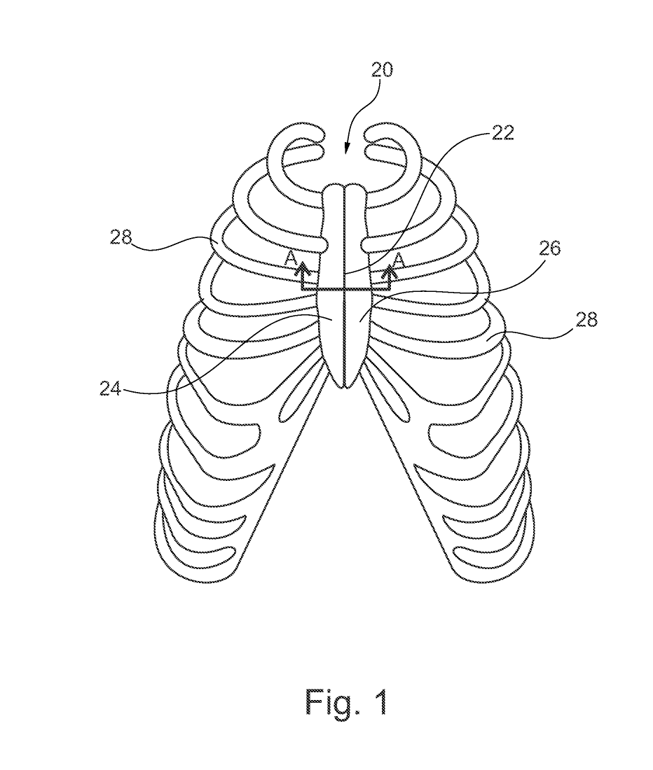

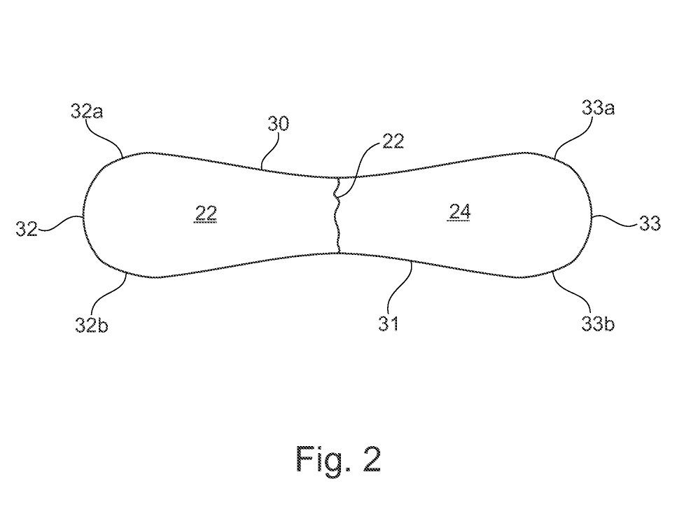

FIG. 2 illustrates a cross-section view along line A-A of FIG. 1 with the sternum having a cut 22 that divides it into the sternum halves 22 and 24. The cross-sectional shape of the sternum generally comprises elongate top and bottom surfaces 30 and 31 that are joined at their ends by first and second bent surfaces 32 and 33. The top and bottom surfaces 30,31 typically possess some curvature as shown in FIG. 2, but generally appear relatively flat in nature as compared to the bent ends 32,33 that possess a large degree of curvature along a short distance. As shown in FIG. 2, in some patient's the top surface 30 may have a generally concave shape and the bottom surface 31 may have a generally convex shape. The top and bottom surfaces 30,31 and first and second ends 32,33 are joined together at corners 32a, 32b, 33a and 33b.

As discussed above, the use of flexible straps to join the split halves of a sternum together are known. However, particularly when the flexible straps are made of a polymeric material, such as polyetheretherketone (PEEK), the thickness of the strap must be large enough to endow the strap with a sufficient amount of strength to bring and maintain the sternum halves in tight abutment without the risk of the strap breaking. To meet this requirement the polymeric straps have a rigidity that inhibits conformability with the cross-sectional shape of the sternum without an ample amount of force being applied to the straps. For this reason, devices like the Sternal ZipFix System™ require the use of special heavy duty applicators to apply such force. Further, the rigidity of such devices makes it difficult to minimize the existence of gaps between the straps and the bone segments they join.

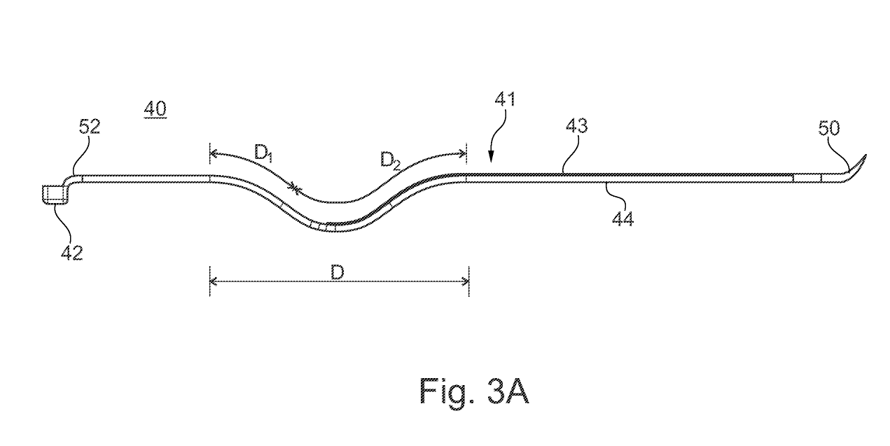

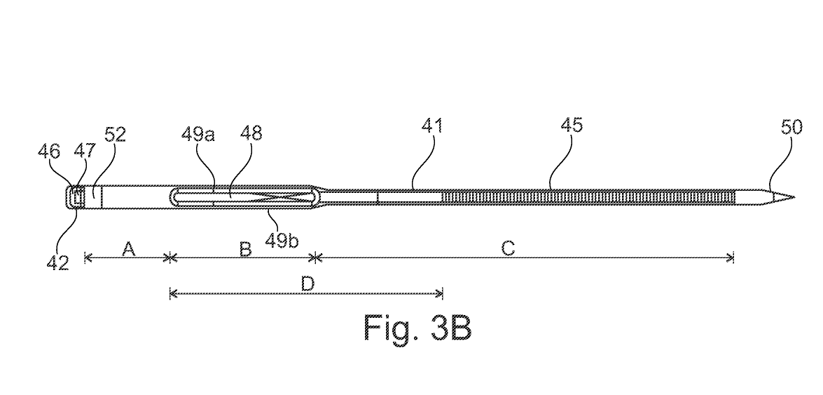

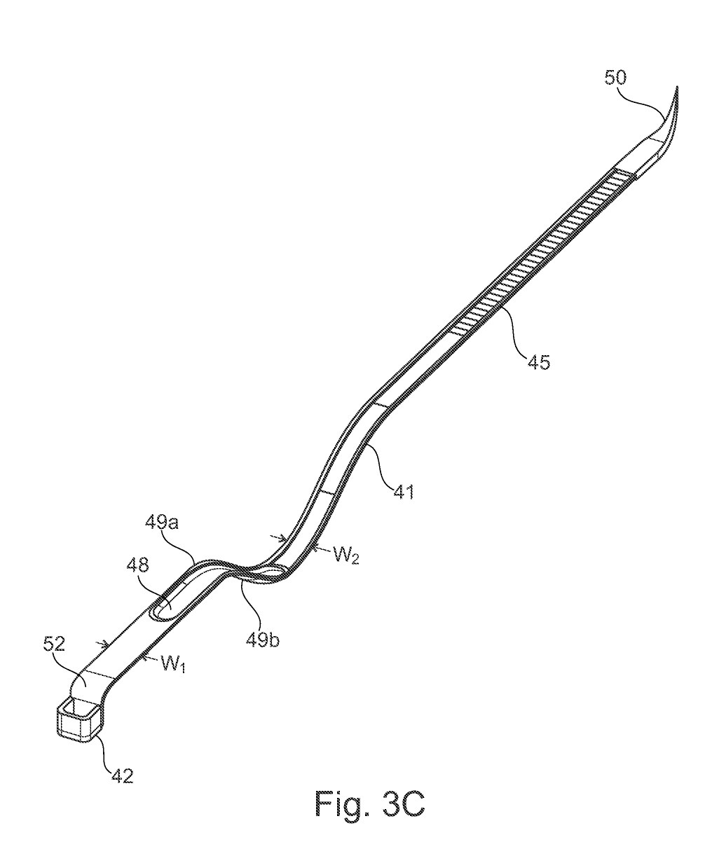

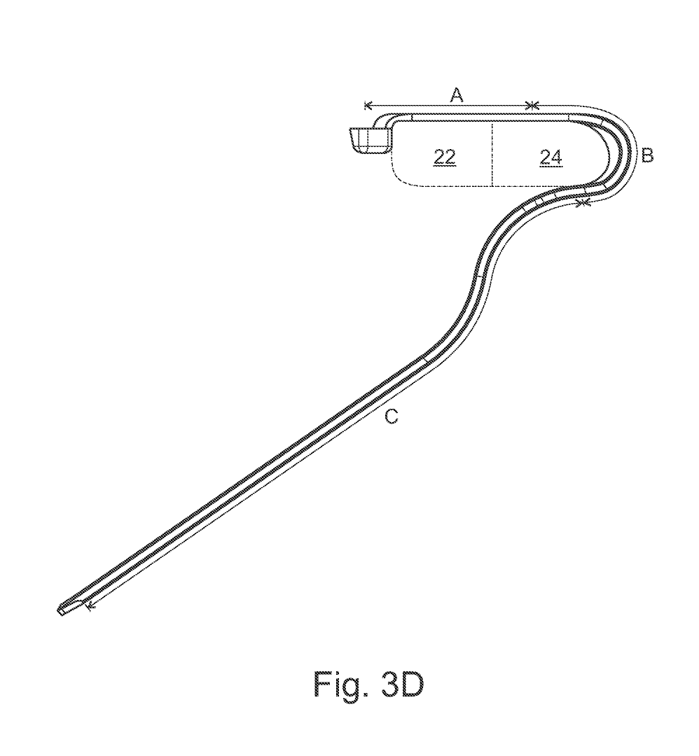

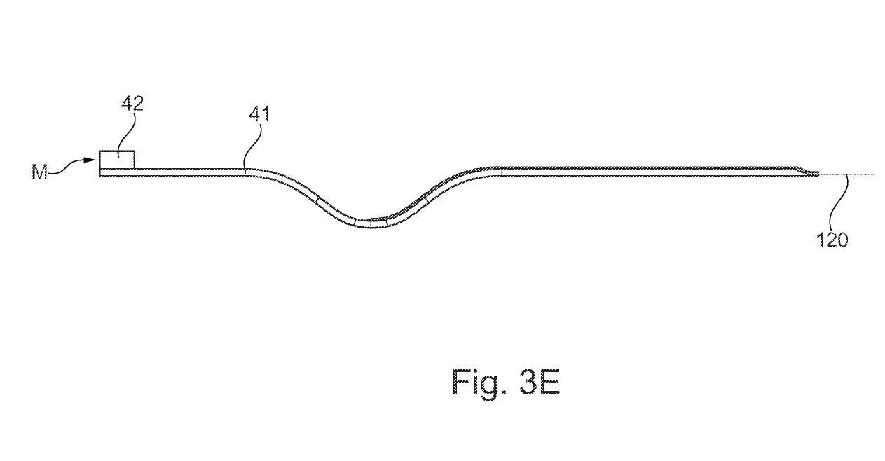

FIGS. 3A-D illustrate various views of a bone fragments fixation device 40 according to one implementation. FIGS. 3A-C show the fixation device 40 in a rest/as-manufactured condition, while FIG. 3D shows the device partially looped about the sternum bone mass 20. The fixation device 40 includes an elongate flexible strap 41 that extends between a locking head 42 at one end and a needle 50 at the opposite end. According to some implementations the flexible strap 41 and locking head 42 comprise a unitary structure moulded from a polymeric material, such as PEEK. The needle 50 is made of a material that is sufficiently rigid to pierce the tissue that surrounds the bone fragments to be joined (such as sternum halves 22 and 24) so that the fixation device 40 may be looped around the bone fragments. According to one implementation the needle 50 is made of a biocompatible metallic material, such as stainless steel. The needle may also be made of other materials such as, for example, polymers, ceramics and composites. The needle 50 may be secured to the strap 41 using any of a variety of conventional means such as bonding, crimping, welding, molding, riveting, etc. As will be discussed in more detail below, upon the fixation device 40 being properly looped about the bone fragments to be joined, the needle 50 is capable of being removed from the strap 41 in order to create a free end of the strap 41. According to some implementations, a needle is not provided at the distal end of the flexible strap 41, the flexible strap in such an instance having a free end.

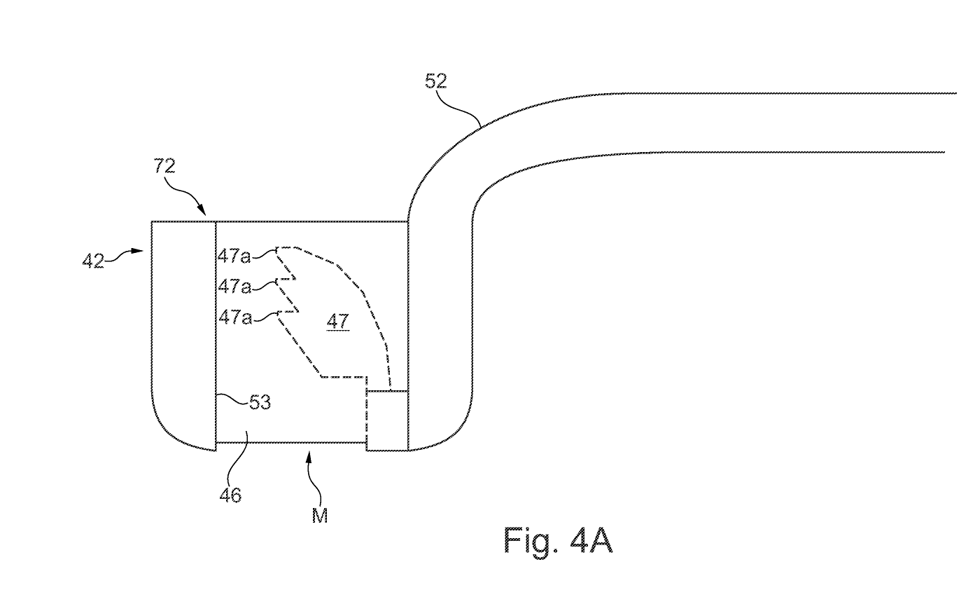

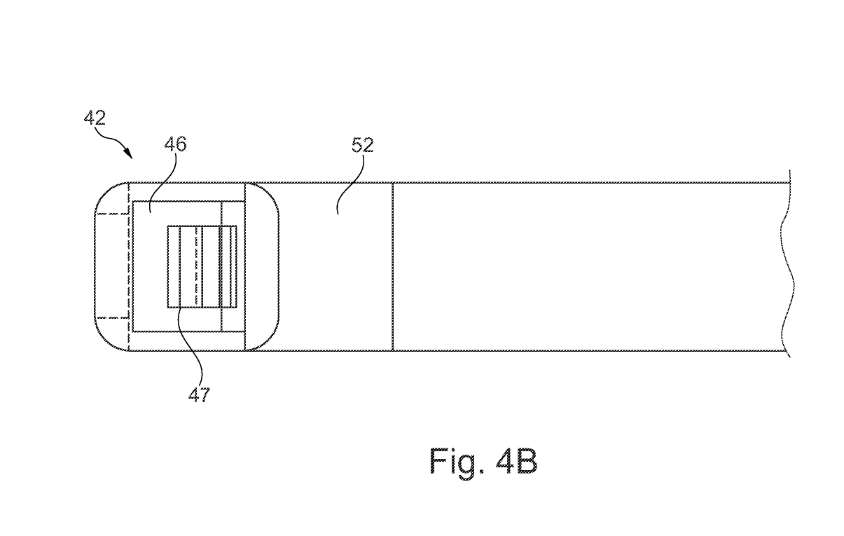

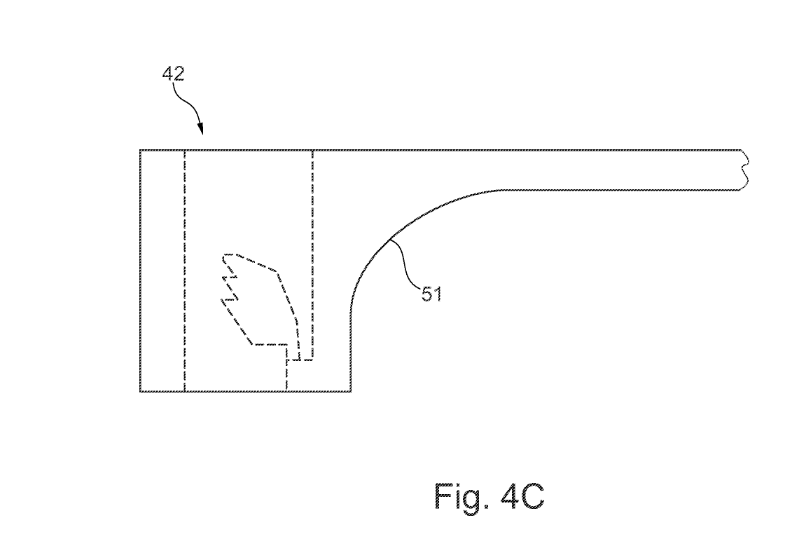

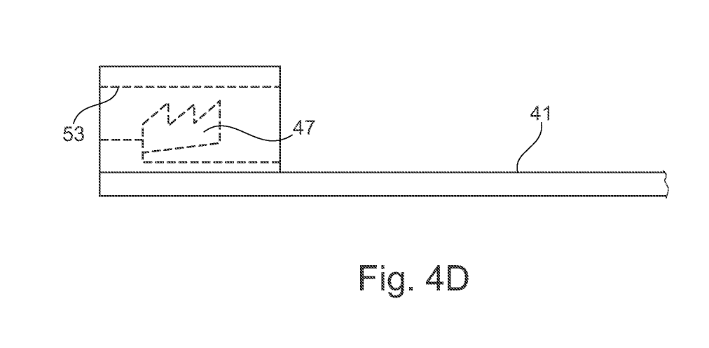

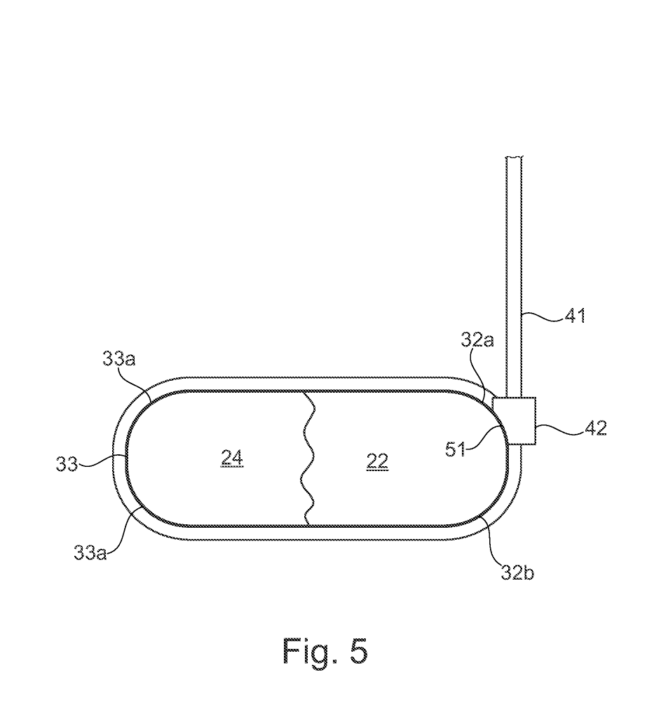

The strap 41 has a top surface 43 and a bottom surface 44 with a plurality of ratchet teeth 45 disposed along an elongate distal portion of the strap. The locking head 42, located at the proximal end of the fixation device 40, defines a transverse channel 46 with a ratchet member 47 situated therein (see also FIGS. 4A and 4B). The ratchet member 47 has one or more teeth 47a adapted to engage the ratchet teeth 45 on the top surface 43 of the strap 41 in a manner that permits movement of the distal portion of the strap 41 through the channel 46 in the direction indicated by the arrow M in FIG. 4A. The distal portion of the strap 41 is adapted to be advanced through the channel 46 of the locking head 42 until the fixation device 40 assumes a looped configuration about the sternum halves 22,24 as shown in the example of FIG. 5. According to some implementations the locking head 42 is arranged with the transverse channel 46 and ratchet member 47 being disposed non-orthogonal to the longitudinal axis 120 of the strap 41, and in some implementations disposed parallel or substantially parallel to the longitudinal axis 120 of the strap 41 as shown in FIGS. 3E and 4D. An advantage of this arrangement is that it provides greater flexibility in the positioning of the locking head 42 when the fixation device 40 is securely tightened about the sternum halves 22,24. In some implementations, the ratchet teeth 45 are disposed on the bottom surface 44 of the strap 41. In such implementations the ratchet member 47 is attached to the wall 53 within the locking head 42 and faces in a direction opposite to that shown in FIG. 4A.

According to some implementations the strap 41 has a proximal elongate portion A, an intermediate elongate portion B and a distal elongate portion C, wherein the intermediate elongate portion B is more flexible than the distal elongate portion C. In other implementations the intermediate elongate portion B is more flexible than each of the proximal and distal elongate portions A and C. According to some implementations, the location and length of the elongate portion B results in the elongate portion B being placed across the entirety of bend 33, including corners 33a and 33b upon the fixation device 40 assuming a looped configuration about the sternum 20. Other configurations are also contemplated, as will be discussed in more detail below.

According to some implementations, as shown in FIGS. 3A and 3B, the intermediate elongate portion B of the strap 41 includes an elongate through opening 48 that extends through the top and bottom surfaces 43 and 44 of the strap 41, the existence of the through opening 48 endowing the intermediate elongate portion B with a greater degree of flexibility than at least the distal elongate portion C, and in some implementations with a greater degree of flexibility than both of the proximal and distal elongate portions A and C. On each side of opening 48 is disposed an elongate strip 49a and 49b, the elongate strips constructed to act together to provide the intermediate elongate portion B with an enhanced level of flexibility with respect to elongate portion C (or elongate portions A and C) while also providing the intermediate elongate portion B with a sufficient amount of strength to withstand the tensile and bending stresses imparted thereon when the fixation device 40 is tightly looped about the sternum 20 to bring the sternum halves 22,24 into tight abutment.

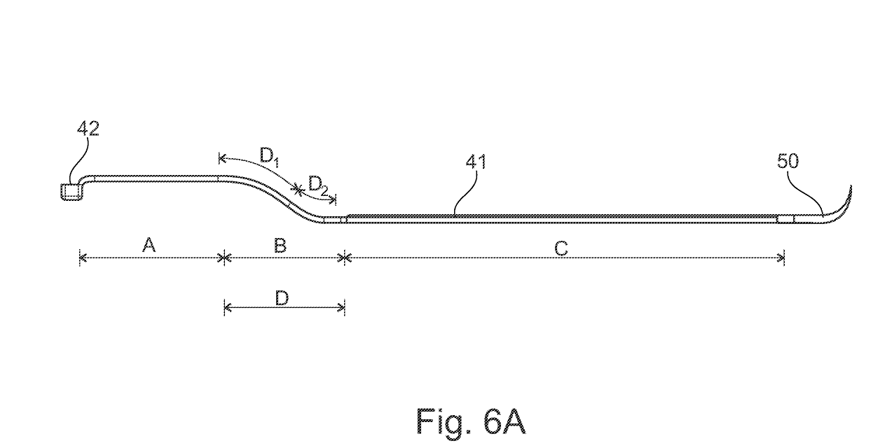

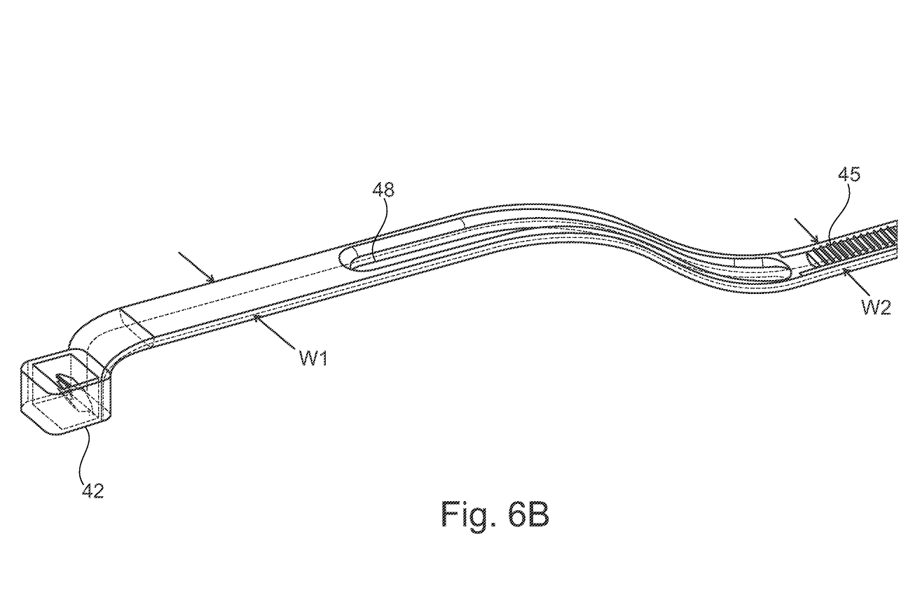

In some implementations, like those shown of FIGS. 3 and 6, the proximal and intermediate elongate portions A and B have a width dimension W1 that is greater than the width dimension W2 of the distal elongate portion C. The greater width dimension of W1 provides several advantages. First, it provides for flexibility in the design of the through opening 48 and strips 49 allowing them to be tailored to meet the requirements associated with fixating a variety of different types of bone fragments. Secondly, it assists in positioning and stabilizing the fixation device 40 during its placement about bone fragments to be joined. For example, as shown in FIG. 3D, when the fixation device 40 is being placed about the sternum halves 22 and 24, the elongate portions A and B of the strap 45 extend across the top surface 30 of the sternum and the bend 33 as the fixation device is initially placed about the sternum halves. The wider footprint of the elongate portions A and B helps in maintaining the fixation device in proper alignment on the sternum 20 during the completion of securing the sternum halves together. Further, as shown in FIGS. 3A-D, in some implementations the proximal elongate portion A includes a bend 52 at its proximal end where it is joined with the locking head 42. The bend 52 may be contoured to rest on the upper corner 32a of the sternum halve 22 (see FIG. 3D). Providing a wider footprint at the bend 52 assists in ensuring a proper placement of the fixation device 40 as it is initially positioned on the sternum 20 and also assists in maintaining the fixation device 40 in a proper angular orientation about the sternum as it is being affixed thereto.

In some implementations, as shown in FIGS. 4C and 5, the locking head 42 has a curved inner side 51 that is also adapted to rest on a portion of the upper corner 32a of sternum halve 22.

In some implementations the fixation device 40 comprises a flexible strap that is straight, or substantially straight, when the fixation device is in the rest/as-manufactured state (not shown in the figures). In other implementations, like those shown in FIGS. 3 and 6, the flexible strap 41 comprises a curved portion D that lies partially or fully within the enhanced flexibility region B of the strap 41. In the implementation of FIG. 3, the curved portion D begins at or near the proximal end of elongate portion B and extends distally to a location at or near the onset of the ratchet teeth 45. As shown in FIG. 3A, in some implementations the curved portion D includes a first segment D1 that has a curvature that is partially or completely complementary to the curvature of the bend 33 of the sternum halve 24. In some implementations the curved portion D also includes a second segment D2 distal to the first segment D1 that is adapted to extend toward the bottom surface 31 of the sternum 20 when the fixation device 40 is initially loosely looped about the sternum halves 22 and 24. According to some implementations the configuration of the curved portion D results in the strap assuming a loop about the sternum halves 22 and 24 that when tightened has a perimeter that is smaller than is otherwise achievable without the existence of the curved portion D. That is, it has an optimized perimeter with respect to the perimeter of the bone cross-section. In the implementation of FIG. 3 the second segment D2 extends across all, or at least a significant portion of the bottom surface 31 of the sternum 30. In the implementation of FIG. 6 the second segment D2 extends across only a portion of the bottom surface 31 of the sternum 30. An inclusion of the first segment D1 assists in initially adapting the fixation device 40 to the sternum 20 and minimizes the existence of gaps between the strap 41 and the sternum 20 after the device assumes its tightly secured state about the sternum halves 22 and 24. An inclusion of the second segment D2 assists in minimizing the existence of gaps between the strap 41 and the bottom surface 31 of the sternum 20 by it extending toward the bottom surface 31 of the sternum 20 as partially shown in FIG. 3D. According to some implementations the configuration of the second segment D2 is selected so that it readily assumes a straight or substantially straight profile when the strap 41 is securely tightened about the sternum halves 22 and 24. As shown in FIGS. 2 and 3D, the bottom surface 31 of the sternum 20 can comprise a curved surface that approximates a convex curve. According to some implementations the configuration of the second segment D2 causes the second segment D2 to at least partially approximate the curvature of the bottom surface 31 of the sternum 20 when the fixation device 40 assumes a secured state about the sternum, to minimize the existence of gaps between the strap 41 and the sternum 20.

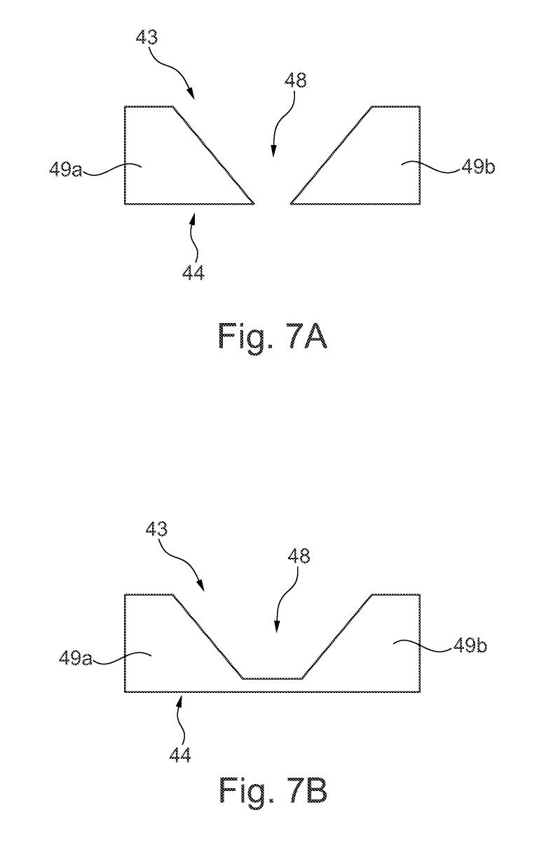

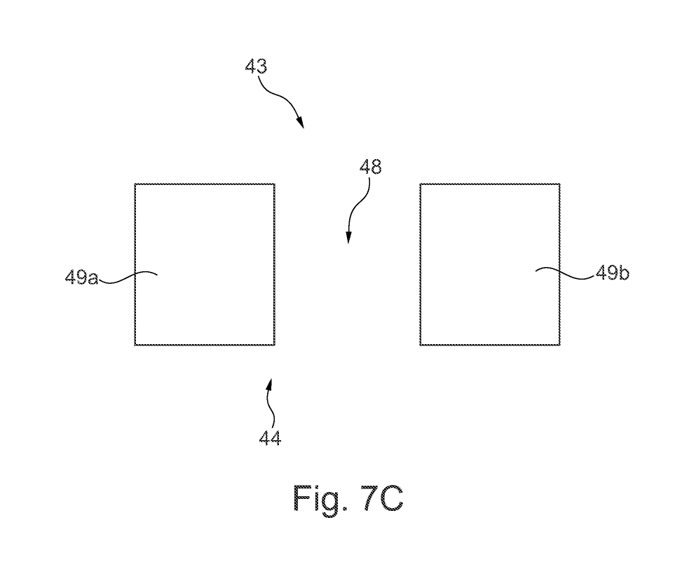

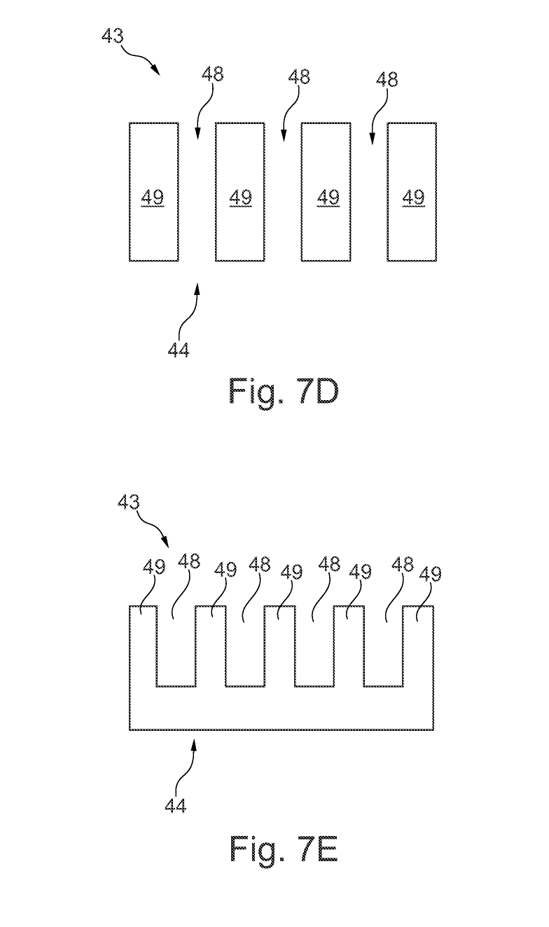

In the fixation devices of FIGS. 3 and 6 a through opening 48 that extends between the top and bottom surfaces 43 and 44 of the strap 45 endow the intermediate elongate portion B of the strap 41 with greater flexibility than the distal elongate portion C or with a greater flexibility of both the proximal and distal elongate portions A and C. In other implementations, like the examples of FIGS. 7B and 7E, openings that extend only partially through the strap 41 are used to achieve, along with other dimensional and material characteristics, a desired flexibility and strength of the intermediate elongate portion B.

FIGS. 7A-E illustrate cross-section views of the intermediate elongate portion B of the strap 41 according to some implementations. FIG. 7A illustrates a cross-section according to what is depicted in the implementations of FIGS. 3 and 6. As shown, as a result of the shape of the opening 48, each of the strips 49a and 49b comprise a trapezoidal cross-section wherewith a larger surface area is provided along the bottom surface 44 of the strap 41 as compared to the top surface 43 of the strap. Such a configuration provides a number of advantages. First, it enables the inclusion of a relatively large opening while still providing the bottom surface 44 of the strap 41 with an area for contacting the outer surface of the sternum 20 that is relatively large. Second, because tensile stresses in the strap 41 are greatest at and near its top surface 43, the smaller cross-section of the strips 49a and 49b at and near the top surface 43 (as compared to the cross-section at and near the bottom surface 44) advantageously contributes most in enhancing the flexibility of the intermediate elongate portion B. For a given opening size, it further maximizes the cross-sectional area of the strips 49a and 49b. By maximizing the cross-sectional area of the strips 49a and 49b, the strips are more easily designed to withstand the tensile and bending stresses imparted upon them when the fixation device 40 is tightly looped about the sternum 20 in the secured state. In the implementation of FIG. 7B the opening 48 is in the form of a recess or channel that extends only partially through the strap 41. In each of the implementations of FIGS. 7A-B, as well as that of FIG. 7E, the large contact area provided at the bottom surface 44 of the strap 41 allows a greater amount of force to be applied to the strap 41 without a risk of having the strips 49a and 49b cutting through the bone mass.

In other implementations the opening 48 and strips 49a and 49b may each comprise rectangular shapes as depicted in FIG. 7C. Multiple through openings 48 or partial openings (e.g. recesses/channels) may also be provided to produce a cross-section comprising a multitude of elongate strips 49 like those shown in FIGS. 7D and 7E. In some implementations a combination of through openings and non-through openings (e.g., recesses/channels) are provided. Further, although not shown in FIGS. 7A-E, the corners formed by the inclusion of the opening(s) in the intermediate elongate portion B may be rounded for the purpose of reducing stress concentrations in said regions.

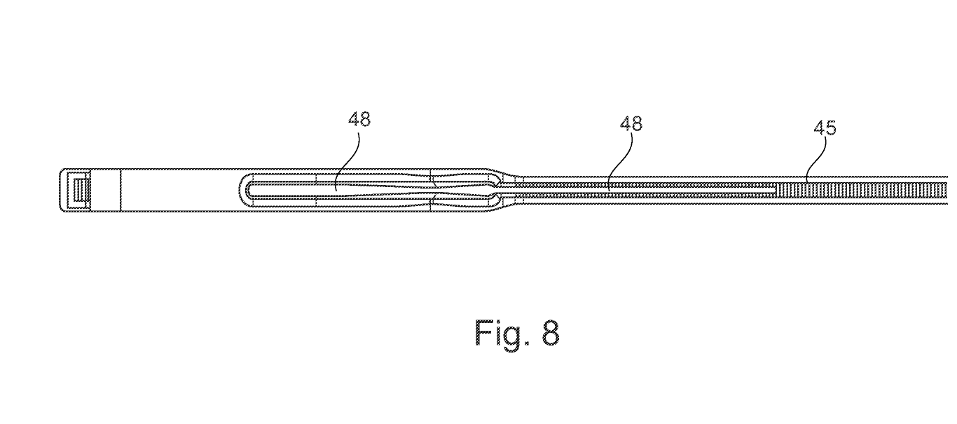

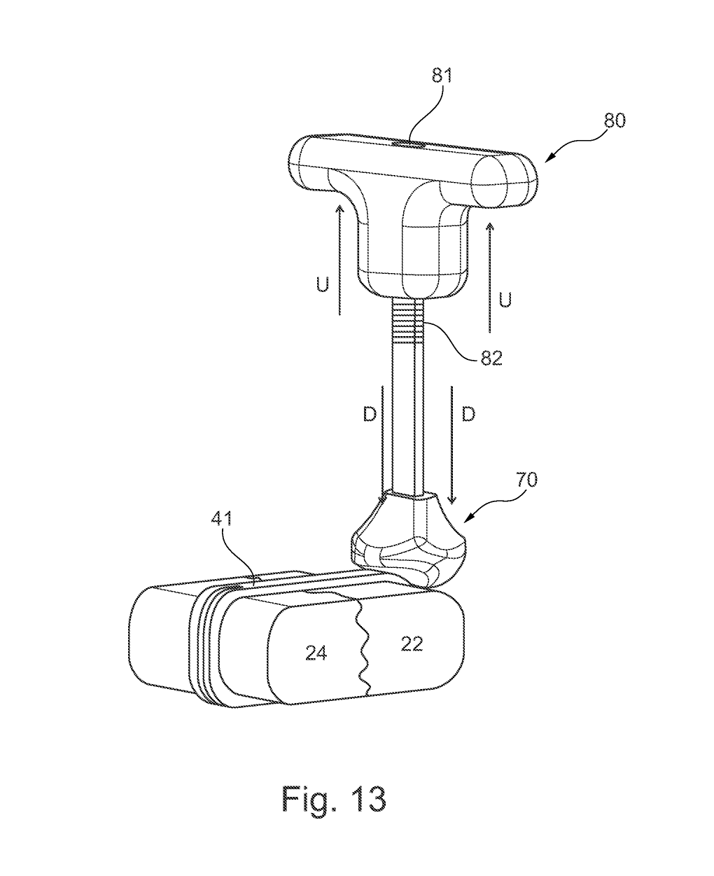

In the implementation of FIG. 3 an opening is provided only within the elongate region B. In other implementations not shown, an elongate opening or recess separate to or contiguous with opening (or recess) 48 may be provided in a region of the strap 41 distal to intermediate elongate portion B. For example, in some implementations an elongate opening or recess may extend distally and terminate proximal to the location of the ratchet teeth 45. In other implementations the opening or recess may extend distally into the ratchet teeth 45. The same holds true for the implementation of FIG. 6 in that an elongate opening or recess separate to or contiguous with opening (or recess) 48 may be provided in a region of the strap 41 distal to intermediate elongate portion B. For example, in some implementations an elongate opening or recess may extend distally into the ratchet teeth 45 as shown in FIG. 8. By extending the length of the region of the strap 41 that possesses an opening or recess the strap 41 is advantageously better able to conform to a greater circumferential area of the sternum 20. For example, as shown in the implementation of FIG. 13, the opening or recess 48 may extend across each of corners 33a, 33b and 32b of the sternum 20. It is important to note that strap 41 may contain a series of spaced-apart openings or recesses in lieu of a single elongate opening. The provision of multiple spaced-apart openings can assist in maintaining the structural integrity of the strap 41 without significantly affecting its flexibility. Moreover, the openings may be strategically placed to provide enhanced flexibility only in designated regions, such as those that are intended to coincide with portions of the bends 32 and 33 of the sternum 20. For example, according to some implementations a first opening is provided that extends across the corners 33a and 33b of bend 33, and a second opening is provided that extends across the corner 32b of bend 32. Other opening/recess configurations are also contemplated.

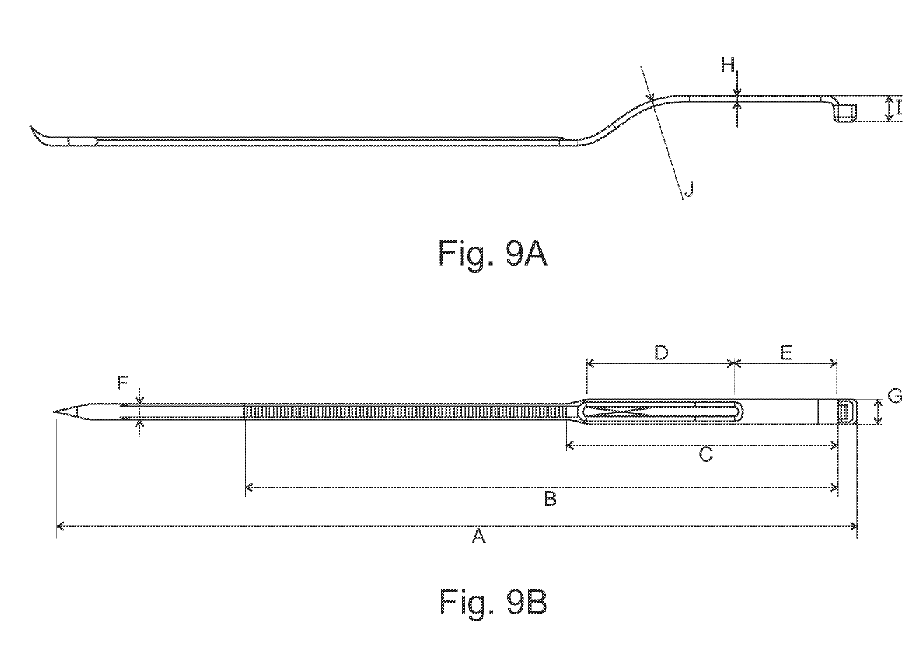

The dimensional characteristics of the fixation devices disclosed and contemplated herein will vary depending upon the type of bone fragments to be joined. In instances of joining together the sternum halves 22,24 in a human body with a fixation device of the implementation of FIG. 6, the fixation device may comprise the dimensional characteristics A-J as illustrated in FIGS. 9A-B. Dimension A may be between about 180 to about 380 millimeters (about 7 to about 15 inches). Dimension B may be greater than or equal to about 150 millimeters (greater than or equal to about 6 inches). Dimension C may be between about 45 to about 55 millimeters (about 1.8 to about 2.2 inches). Dimension D may be between about 20 to about 35 millimeters (about 0.8 to about 1.4 inches). Dimension E may be between about 20 to about 25 millimeters (about 0.8 to about 1.0 inches). Dimension F may be between about 3.5 to about 4.5 millimeters (about 0.138 to about 0.183 inches). Dimension G may be between about 3.5 to about 5.0 millimeters (about 0.138 to about 0.196 inches). Dimension H may be between about 0.7 to about 1.2 millimeters (about 0.03 to about 0.047 inches). Dimension I may be between about 5.5 millimeters (about 0.22 inches). Dimension J may be a radius of curvature of about 40 millimeters or less (about 1.6 inches or less).

As discussed, upon the fixation device 40 being properly looped about the bone fragments to be joined, the needle 50 (if present) is removed from the flexible strap 41 in order to create a free end of the strap 41. The free end of the strap 41 is adapted for being inserted into the channel 46 of the locking head 42 in a direction indicated by arrow M in FIG. 4A. At a point in time when the distal free end of the strap 41 transverses the channel 46 of the locking head 42, the ratchet teeth 45 on the top surface 43 of the strap 41 come into engagement with the teeth 47a of the ratchet member 47 of the locking head 42. Upon the ratchet teeth 45 of the strap 41 engaging with the teeth 47a of the ratchet member 47, movement of the strap 41 in a direction opposite to that indicated by the arrow M in FIG. 4A is prevented. Upon the free end of the strap 41 emerging from the topside 72 of the locking head 42, as shown, for example, in FIG. 5, the portion of the strap 41 that protrudes from the topside of the locking head may be acted upon to pull a remaining portion of the strap 41 through the locking head 42 until the bone fragments to be secured are in tight abutment with one another, as shown, for example, in FIG. 5. As discussed above, by virtue of the inclusion of areas of enhanced flexibility (such as intermediate portion B) and/or the inclusion of bend portion D within the fixation device 40, in some instances an amount of force effective to secure the bone fragments together may be achieved by simply gripping and pulling on a distal portion of the strap 41 with a human hand or with the use of a standard surgical tool, such as surgical forceps. According to some implementations, such as when the strap 41 is made of PEEK, the length of strap 41 protruding from the topside surface of the locking head 42 may be severed from the fixation device 40 by simply bending the strap 41 back and forth along the topside surface of the locking head until it breaks. Other methods of severing the excess length of the strap 41 may also be used such as a cutting by use of scissors or other type of cutting tool.

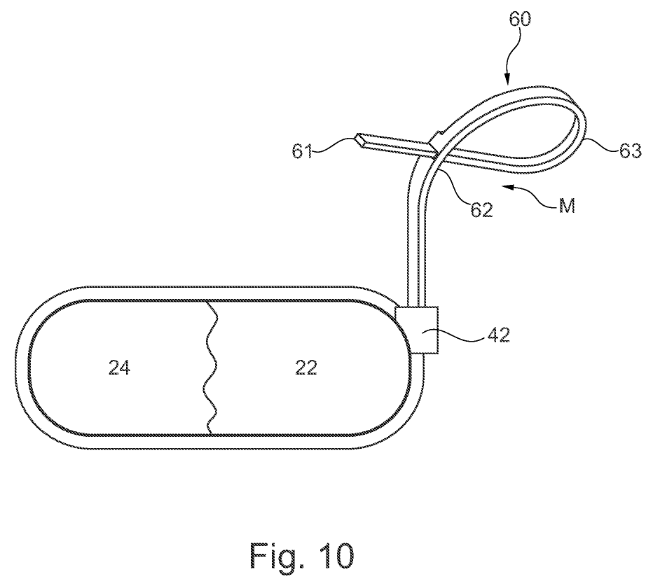

According to some implementations, as shown in FIG. 10, a distal portion of the strap 41 is equipped with features that permit the formation of a loop 60 after the distal portion has been passed through the locking head 42. According to some implementations, the loop 60 is formed by passing the free end 61 of the strap 41 through an opening 62 in the strap as shown in FIG. 10. The loop 60 advantageously provides the surgeon with means for securely gripping the distal end portion of the fixation device by inserting one or more fingers through the loop. This enables the surgeon to apply a greater amount of tension to tighten the strap 41 about the bone fragments 22 and 24 than is otherwise possible without the existence of the loop 62.

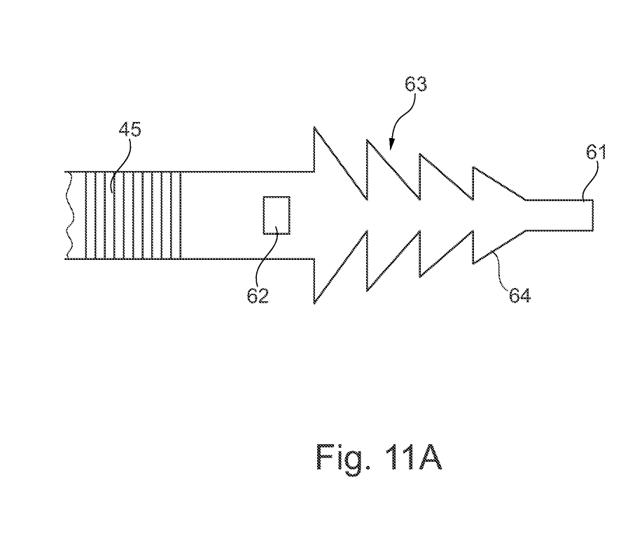

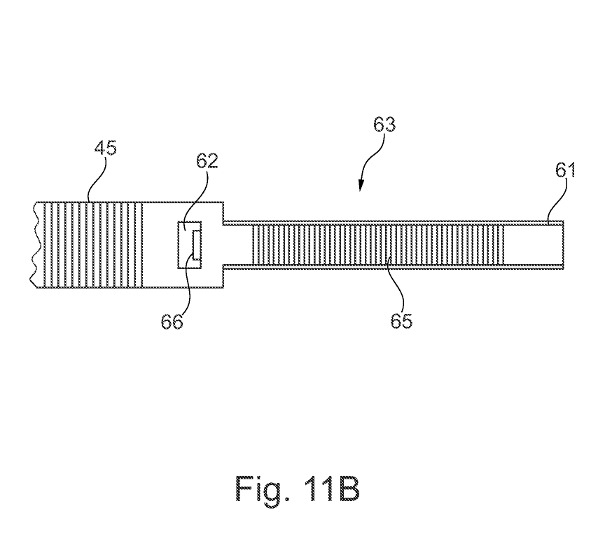

According to some implementations the distal end portion 63 of the strap 41 possesses one or more features that are adapted to engage with a tab in the opening 62 and/or the side walls of the opening 62 so that advancement of the distal end portion 63 through the opening 62 is permitted only in the direction indicated by the arrow M in FIG. 10. According to one implementation, the distal end portion 63 comprises a set of flat, flexible and angular wings 64 that are serially disposed along its length as shown in FIG. 11A. In practice, upon the fixation device 40 being looped bout the bone fragments 22 and 24 with the distal end portion 63 having passed through the locking head 42, the loop 60 is formed by passing at least a one of the angular wings 64 through the opening 62. The flexible nature of the wings 64 permit them to be passed through the opening in the direction of arrow M. Upon having passed through the opening 62, the wings 64 at least partially or fully assume their original shape to prevent, or otherwise inhibit, a movement of the distal end portion 63 in a direction opposite to that indicated by the arrow M once the loop 60 is formed. FIG. 11B shows another implementation wherein ratchet teeth 65 provided on a surface of the distal end portion 63 are adapted to engage with a tab 66 within the opening 62 to prevent, or otherwise inhibit, a movement of the distal end portion 63 in a direction opposite to that indicated by the arrow M once the loop 60 is formed.

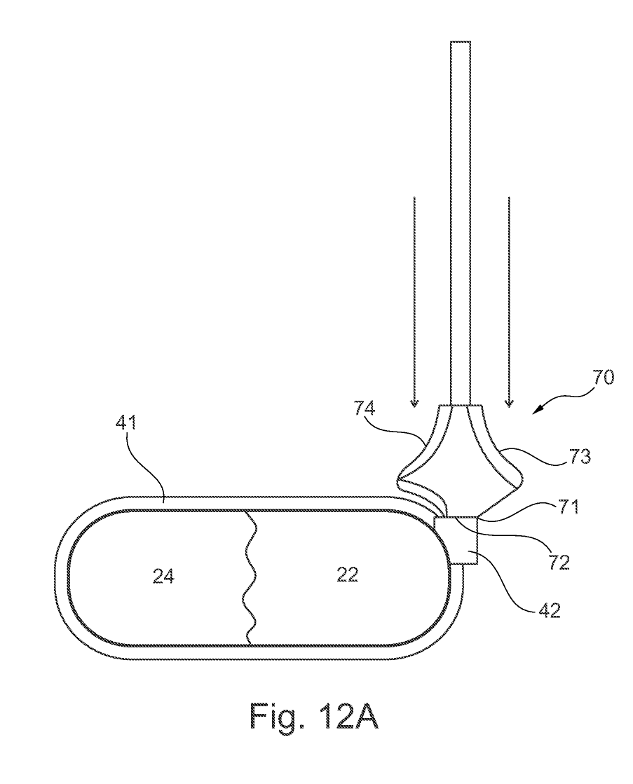

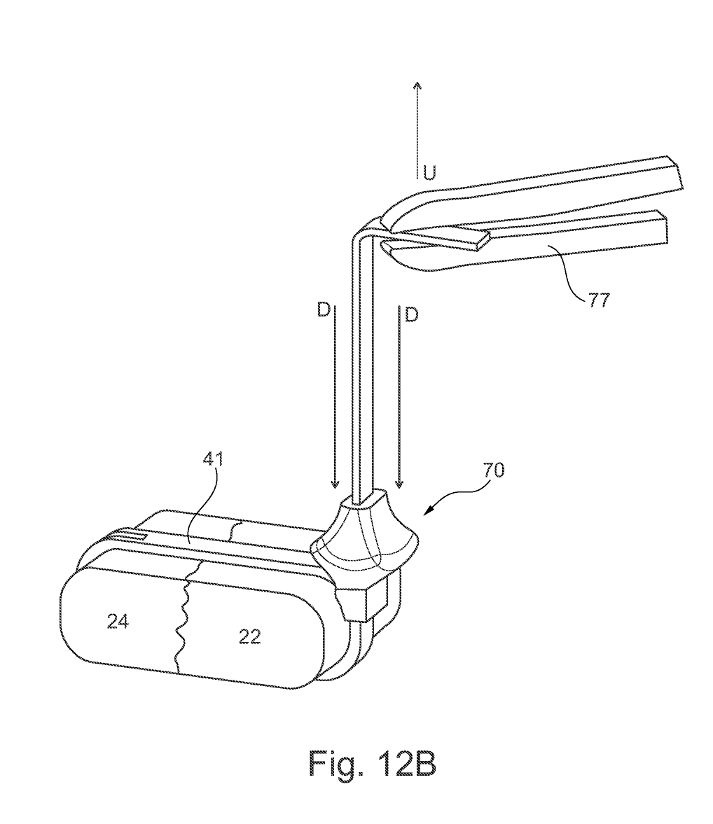

According to other implementations, a locking head force applicator 70 is provided, as shown in FIG. 12A, that has a bottom surface 71 adapted to rest against the topside surface 72 of the locking head 42. According to one implementation, the applicator 70 has first and second surfaces 73 and 74 that are adapted for being acted upon by the thumb and index finger of a surgeon engaged in the operation of securing together bone fragments 22 and 24. The applicator 70 has a through opening (not shown) through which freely passes the distal end portion of the strap 41. In use, upon the final stages of tightly tensioning the strap 41 about the bone fragments 22 and 24, the applicator 70 is passed over the distal end portion of the strap 41 until the bottom surface 71 rests against the topside surface 72 of the locking head 42 as shown in FIG. 12A. Thereafter, the surgeon may grip the distal end portion of the strap 41 with the use of a hand or with a gripping tool, such as forceps 77 (as shown in FIG. 12B) while holding the applicator 70 against the topside surface 72 of the locking head 42. The surgeon then, while applying an upward force U by the use of only a hand or with the use of standard surgical forceps 77 as shown in FIG. 12B, may also apply a downward force D on the applicator 70 to effectuate a secure tightening of the strap 41 about the bone fragments 22 and 24. The use of the applicator 70 enables the surgeon to apply a greater amount of tension to tighten the strap 41 about the bone fragments 22 and 24 than is otherwise possible without the use of the applicator. The applicator 70 also advantageously provides a platform for stabilizing the position of the fixation device 40 as tensioning forces are being applied to it.

According to other implementations a force applicator 80 adapted for being coupled with the distal end portion of the strap 41 is provided as shown in FIG. 13. In the implementation of FIG. 13, the applicator 80 possesses a through opening 81 for receiving the distal end portion of the strap 41. In the implementation of FIG. 13 the applicator is T-shaped so as to provide surfaces that enable it to be firmly gripped by the hand of a surgeon. According to some implementations, the distal end portion of the strap 41 includes ratchet teeth 82 that are adapted to engage a ratchet member (not shown) disposed within the through opening 81. The ratchet member and ratchet teeth 82 are configured to permit the distal end portion of the strap to be advanced only in an upward direction (as viewed in FIG. 13). Upon there being an engagement of the ratchet teeth 82 with the ratchet member of the applicator 80, the applicator 80 may be gripped with the hand of the surgeon and pulled upward as indicated by the arrows U in FIG. 13 to effectuate a secure tightening of the bone fragments 22 and 24. According to some implementations, the applicator 70 may be used in conjunction with the applicator 80 to apply the tension for securing the bone fragments tightly together.

According to some implementations, the applicators 70 and 80 are made of a polymeric material, such as, for example PEEK. It is appreciated, however, that the applicators may be made of any of a variety of materials. It is also important to note that the shape of the applicators 70 and 80 may vary and are not limited to the shapes depicted in the accompanying figures. Applicator 80 may, for example, take the form of an L-shape, spherical or semi-spherical shape or any other shape capable of being gripped by the human hand.

According to some implementations a kit is provided that includes a requisite number of fixation devices 40 necessary for properly securing the target bone fragments together. According to some implementations, the kit includes one or more fixation devices 40 and an applicator 70. According to some implementations, the kit includes one or more fixation devices 40 and an applicator 80. In yet other implementations, the kit includes one or more fixation devices 40 and both of applicators 70 and 80. According to some implementations the applicators 70 and 80 are trash disposable after their use with there being no need to employ post-operative sterilization procedures.

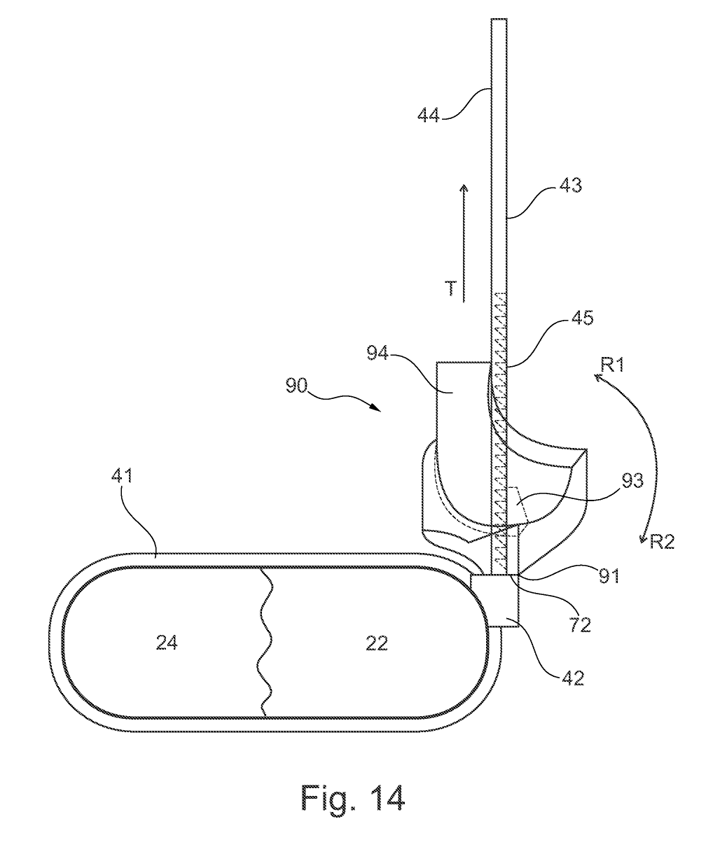

FIG. 14 illustrates a partial cross-section view of a tensioning apparatus 90 useful in inducing an incremental advancement of the distal portion of the strap 41 in the direction of the arrow T once the strap 41 has been initially looped about the bone fragments as depicted. The tensioning apparatus 90 may take an outer form similar to that of the applicator 70 previously disclosed herein. As with applicator 70, apparatus 90 has a bottom surface 91 adapted to abut against the topside surface 72 of the locking head 42 of fixation device 40. The locking head 42 includes a ratchet member that interacts with ratchet teeth disposed on the upper surface of the strap 41 in a manner as previously disclosed. The apparatus 90 is adapted to receive and act upon the distal portion of the strap 41 to induce a tensioning of the strap as the apparatus is rotated back and forth in the directions indicated by arrows R1 and R2. Within a through opening of the apparatus 90 through which the distal portion of the strap 41 is permitted to pass is a ratchet member 93 configured to engage with ratchet teeth 95 on the strap 41 as shown in FIG. 14. According to one implementation, the ratchet member 93 acts on one the top surface 43 of the strap 41 while a wall segment 94 of the apparatus 90 acts upon the bottom surface 44 of the strap. In use when the distal portion of the strap 41 has assumed a position as shown in FIG. 14, while firmly maintaining the bottom surface 44 of the strap against the wall 94, a rotation of the apparatus 90 in the direction of R1 causes an incremental advancement of the strap through both the tensioning apparatus 90 and the locking head 42. The lever arm provided by wall 94 enables, by virtue of the rotational movement of the tensioning apparatus 90, a larger tension force to be applied to the strap 41 than would otherwise be possible absent the presence of the apparatus acting on the distal portion of the strap. The ratchet member 93 of apparatus 90 acts in the same way as the ratchet member 47 of the locking head 42 in that it functions to permit movement of the strap in a single direction, the direction of arrow T. After having been rotated in the direction R1 to induce a distal advancement of the strap through the locking head 42 and apparatus 90, the apparatus 90 is rotated in the direction R2 to assume a rest position as depicted in FIG. 14. The apparatus 90 may be repeatedly rotated in the afore-described manner until a desired tensioning of the strap 41 is achieved, the desired tensioning being that amount sufficient to bring the bone fragments 22 and 24 into tight abutment with one another.

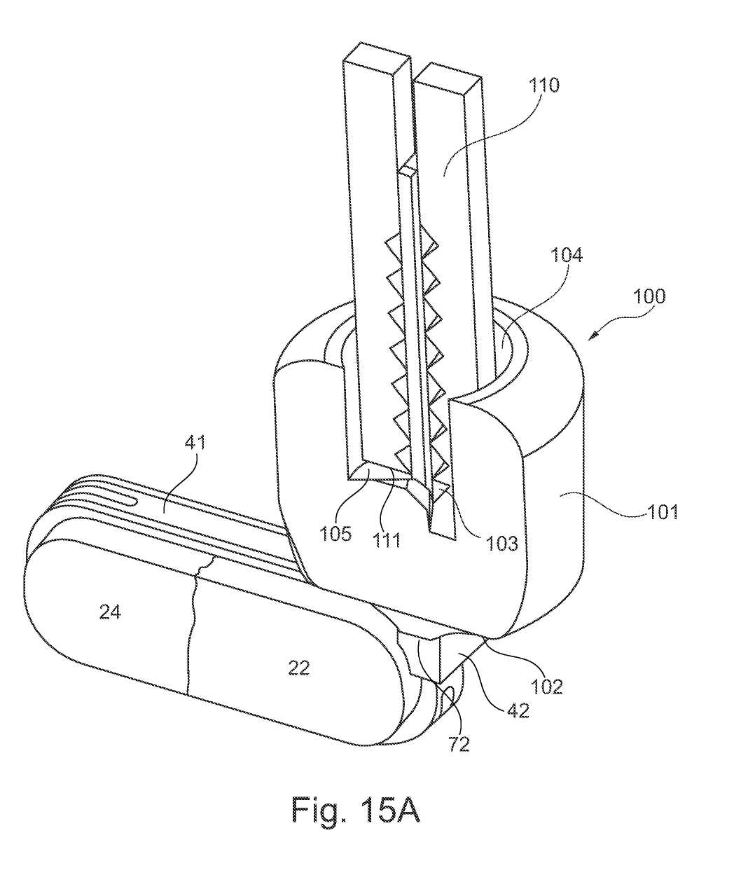

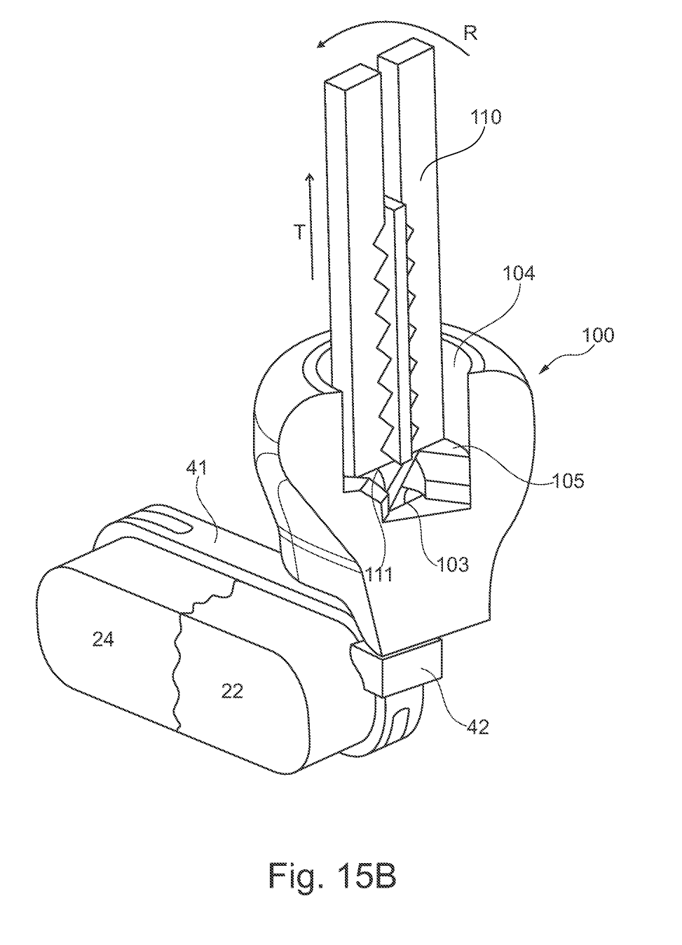

FIG. 15 illustrates another tool and method of incrementally inducing a distal advancement of the strap 41 through the locking head 42 in order to achieve a desired tensioning of the strap. According to one implementation, the tool 100 (shown in partial cross-section) comprises a body 101 having a bottom surface 102 for resting against the topside surface 72 of the locking head 42. The body 101 includes a first opening 103 that passes through a bottom portion of the body, the opening 103 communicating with a larger opening 104 located in a top portion of the body, there being a force bearing surface 105 situated at the junction of openings 103 and 104. In use, when the distal portion of the strap 41 has assumed a position as shown in FIG. 15A, a gripping tool 110 is inserted into opening 104 so that an end surface 111 of the gripping tool 110 abuts the force bearing surface 105. While in this ready position the gripping tool 110 is made to firmly grip the distal portion of the strap as shown in FIG. 15A. Thereafter, while the strap is being firmly gripped and the end surface 111 is in abutment with surface 105, the gripping tool 110 is rotated in the direction of arrow R, as shown in FIG. 15B. As a result of the rotation the strap 41 is caused to incrementally advance distally through the locking head 42 in the direction of arrow T. According to one implementation the gripping tool 110 is rotated about 90 degrees to effectuate the distal advancement. It is appreciated that angular rotations other than 90 degrees may also be employed. Upon the strap being appropriately rotated to cause an incremental advancement of the strap through the locking head 42 by an amount equal to at least a distance between adjacent ratchet teeth 45, the strap is released from gripping tool 110 to permit it to rotate back to its original angular orientation. To the extent additional tensioning of the strap 41 is desired, the gripping and rotating steps are repeated until the desired amount of tension is achieved.

In the implementations disclosed above with respect to FIGS. 3-15, the manner of maintaining the fixation device 40 in a looped configuration is accomplished with the use of teeth 45 disposed along a length of the strap 41 that interface with a ratchet member 47 located within the locking head 42. According to other implementations, not shown in the figures, means other than teeth on the strap cooperating with a ratchet member in the locking head are employed to facilitate the formation and maintaining of the fixation device in a looped configuration. One example includes the use of rings disposed at a proximal end of the fixation device 40 wherewith a portion of the strap 41 is interwoven with the rings in a manner that permits the strap to be advanced in a forward direction through the rings while at the same time preventing, or otherwise inhibiting, a backward movement of the strap through the rings. Other examples include the use of protuberances, or other features, disposed at the proximal end of the fixation device 40, either on the strap itself or within a locking head, that are adapted to engage with other features on the strap to effectuate the formation and maintenance of a loop.

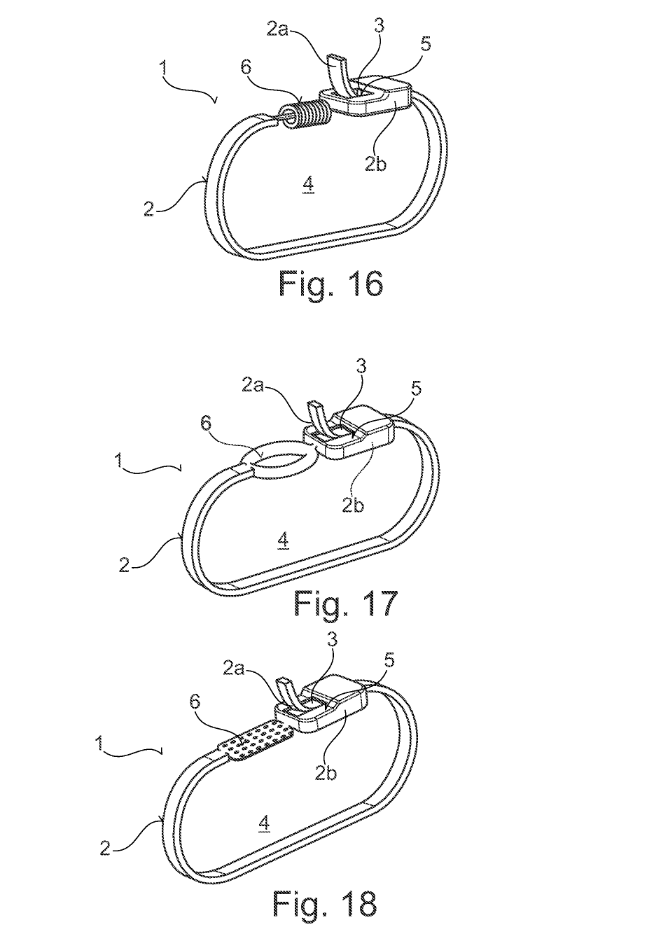

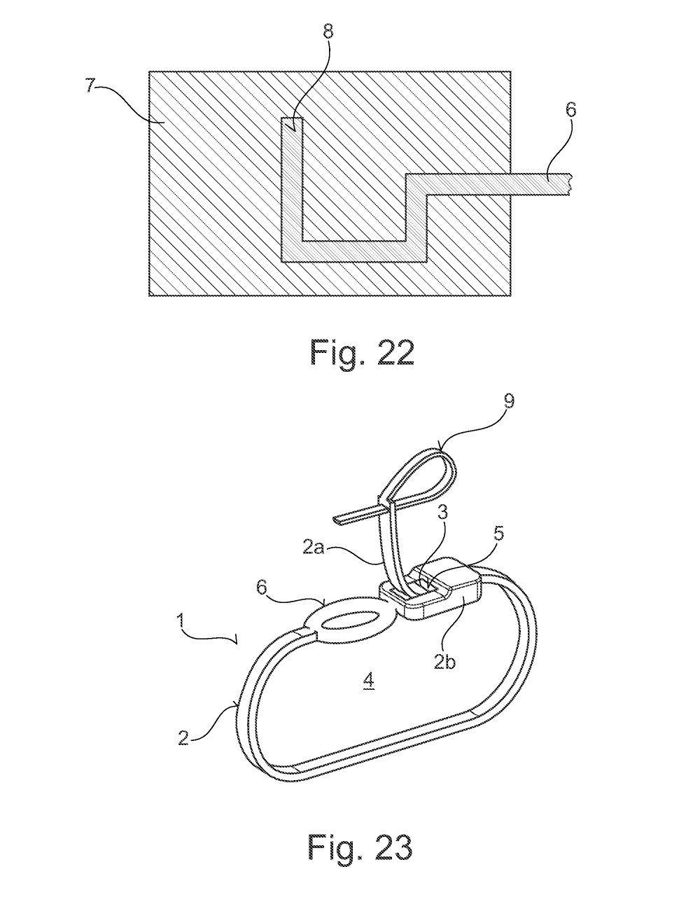

FIGS. 16-24 depict fixation devices according to a variety of other implementations. According to some implementations the devices consists of an oblong element, of biocompatible material and of a sufficient length to loop round aligned bone fragments, such as the bone fragments of the sternum bone, and are provided with an adapted passage to be able to insert one end of the oblong element through it to form a slip loop, the passage and oblong element being provided complementary means of retention that prevent the oblong element from slipping through the passage in the opposite direction to that of insertion.

According to some implementations that oblong element consists of two portions or longitudinal parts of a different nature, one of them being an active strip of a material with a pseudoelastic alloy, preferably superelastic, the configuration of which prevents it passing through the means of insertion, so the loop formed is liable to display a first perimeter L1 temporarily and during its implementation around the bone fragments when the active strip is deformed; and a second perimeter L2 when the active strip recovers its nominal shape either due to disappearance of an effort applied to it, or due to a thermal effort or both, fulfilling the condition that L1>L2. This preferably occurring without further slipping of the oblong element through the passage once the loop is formed.

Pseudo elasticity is known as the situation in which major deformations are obtained by increasing the load that may be recovered when discharged at a constant temperature. Superelasticity, as well as what is known as the rubber effect, are particular cases of pseudoelasticity.

According to some implementations the active strip is a material with a memory thermal effect providing the possibility of that stretch to recover its nominal shape after effort is applied at low temperature, when that active stretch is heated up again.

In any event and under advantageous terms, the lack of tension that may be applied to the loop due to the tightening being applied manually may compensate the reduction of the perimeter of the loop formed when the active strip recovers its nominal shape.

According to some implementations the fixation devices combine materials of a different nature and not all the device is comprised of the active material, for example with shape memory. A device formed fully with material with memory would cause a variation in the perimeter of the loop formed in an uncontrolled manner, as its perimeter during its formation varies considerably according to the parameters of the patient. In other words, the absolute variation in length of the perimeter would vary significantly according to the length of the loop formed.

Providing a device with one or several active strips with a preset length, that shall be the same regardless of the length of the loop formed, it allows one to select optimum retraction values taking into account the statistical perimeter that may have the loop formed for a specific use. In other words, it allows one to select retraction values that make it possible for a same device to adequately fulfil its function for a wide spectrum of population for a specific use, such as repairing a sternum after a sternotomy.

According to some implementations the active strip has the property of retracting between 2 mm and 8 mm on going from L to l. More preferentially, to retract between 3.5 and 6.5 mm to repair the bone tissue of the sternum.

In some implementations the active strip is connected by at least one of its ends to a longitudinal strip of the oblong element that is formed by a mouldable material. According to some implementations the active strip includes means that permit it to be anchored in the mouldable material for safe connection to it.

According to some implementations the shape of the active strip or strips is selected so a loop formed and subject to manual tightening around a bone tissue generates a torque exceeding 200 N so that strip recovers its nominal shape.

According to some implementations an end of the oblong element is adapted to be bent and linked on top of itself to form a pulling grip.

According to some implementations, to take advantage of the pseudo elastic or superelastic properties of the active strip, the device includes a component that exerts a force on the active strip to keep it stretched, that component being adapted to be manipulated in order for it to cease to act on the active strip once the loop is formed.

FIGS. 16-18 illustrate fixation devices according to some implementations. In all of these, device 1 is formed by an oblong element 2 in strip form, in which two ends, 2a and 2b, are distinguished. A protrusion is formed at end 2a of the oblong element 2, preferably with a flat supporting surface to which reference shall be made later on, equipped with an orifice 3, assigned to receive the insertion of opposing end 2a of the oblong element to form a the loop 4. FIGS. 1 to 3 show device 1 with the loop 4 already formed.

The passage 3 and the oblong element 2 are equipped with complementary means of retention 5, that are not displayed in detail (as they are known) that prevent the oblong element 2 from slipping through passage 3 in the opposite direction to that of insertion, so the loop 4 formed cannot be undone. As an example, the oblong element may be provided with retaining teeth on one of its faces, passage 3 having a similar section to the portion of the oblong element 2 that will go through it, equipped with a flexible tongue inside that shall act as a ratchet on the toothed part of the oblong element.

The protrusion of end 2b of the oblong element 2 has a surface, when loop 4 is formed, that faces the inside of the flat the loop 4, to favour its support against the bone tissue to be restored, that shall be bound or tied firmly by device 1.

Unlike known devices, the oblong element 2 of the devices in FIGS. 16-18 include portions or longitudinal strips of a diverse nature, attached or linked together, each one of which forms an active strip 6 that has the virtue of acting when there is a temperature change. Active strip 6 is of a material with thermal memory effect, with superelastic properties, and its configuration prevents it slipping through passage 3 to guarantee that it shall always be placed on the portion of the oblong element 2 looped so as to form the loop 4.

In the examples illustrated, the active strip 6 is located in a zone adjoining end 2b of the oblong element, between two stretches 7 of a mouldable plastic material, that is flexible, not elastically deformable, of which one forms the widening in which passage 3 is located, and the other is extended to end 2a of the oblong element 2, which is foreseen to have a slightly curved shape.

The term thermal memory effect refers to the capacity of the material, when submitted to heating, to recover from the plastic deformation to which it may be submitted under low temperature conditions.

According to some implementations the active strip 6 is made of an alloy known commercially as nitinol, that is an equiatomic alloy of Nickel-Titanium, that has the effect bi-directionally, which means that at certain temperature, it has a first shape that may change upon a change in temperature, but if the first temperature is recovered, the first shape is also recovered. This is achieved by thermo-conforming the material for each shape at each temperature.

According to some implementations, starting from a nominal shape, the active strip 6 is deformed at a temperature between 0° C. and 5° C. stretching it; device 1 is applied on the patient's body, forming the loop 4 around the bone tissue to be repaired, for example around the sternum after a sternotomy, and naturally, at body temperature, the active strip 6 retracts, that is, it goes back to its nominal shape, that is shorter, thus causing a reduction in the perimeter of the loop 4 formed. That reduction allows, on one hand, compensation of the insufficient tension that the surgeon may transmit on forming the loop 4 without using specific instruments for the purpose, and on the other hand, and due to the superelastic property of the nitinol, small expansions may take place in the bone tissue to repair it by the loop 4 recovering its working tension again.

In the variant of FIG. 16, the active strip 6 is configured in a spring shape. More specifically, it has the shape of a filiform element in a cylindrical helix. This spring shaped geometry allows major elastic stretching of the active strip 6 without suffering plastic deformation. The force exerted by the spring may be directly proportional to the deformation applied.

In the variation of FIG. 17, the active strip 6 has a ring shape. The ring-shaped geometry allows a spring effect to be generated with an essentially flat profile. The regularity of the geometry minimizes the concentrations of tensions and thus the weak points and allows a folded and stretched position to be maintained externally, that may be released and folded to the nominal shape by activation of a simple mechanism if necessary.

The displacement values obtained at an equal length of the active strip 6 are lower than those with a spring shape, but the strength values obtained have been similar.

In the variation in FIG. 18, the active strip 6 is configured in sheet form. More specifically, it is formed by a sheet of nitinol equipped with a series of perforations distributed regularly over its surface. In the example, the perforations are aligned, forming various rows that run in an essentially transversal direction to the longitudinal axis of the oblong element 2. In each row, the distance that separates two consecutive perforations remains constant.

The plate shaped geometry allows a greater effort to be generated with an essentially flat shape. The mechanism to allow the geometry to superelastically recover its nominal shape, if necessary, may be very simple. The levels of deformation that are obtained at an equal length of the active strip 6 are lower than the preceding two variations, although the force exerted is greater.

According to some implementations, the dimensions of the possible geometric forms are selected so the active strip 6 retracts a distance between 2 mm and 8 mm, which would mean a reduction in the perimeter with a the loop 4 that forms the binding of a sternum between 2.5% and 10%, according to the size of the sternum.

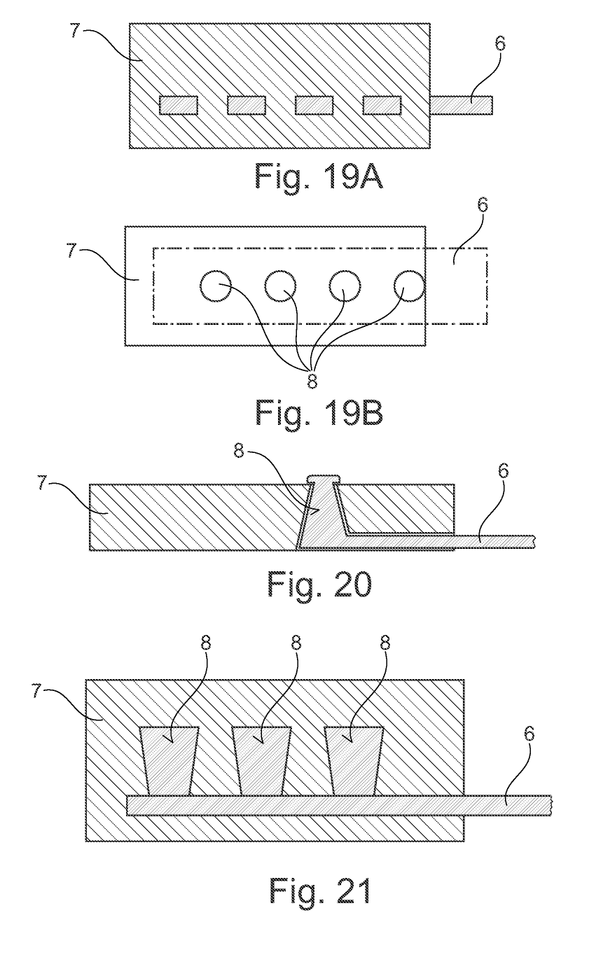

FIGS. 19A-B show schematic views of the longitudinal section and plan view, respectively, so as to link or join the active strip 6 of a device 1 with an adjoining strip of the same oblong element 2 that forms the device 1. The end of the active strip 8 is configured in a plate shape and is equipped with anchorage means 8, formed in the example by passing orifices, that are embedded in the plastic material of which that stretch 7 is constituted, adjoining the oblong element 2. This is achieved by overmolding the plastic around the end of the active strip 6 equipped with anchorage means 8.

FIGS. 20-22 show alternatives for a firm, non-separable union of the active strip 6 of a device 1 to a longitudinal strip 7 of the oblong element 2 that runs next to it.

FIG. 23 represents a device 1 in which the oblong element 2, specifically its end 2a, is adapted to bend and loop on itself to form a pulling grip 9 that shall facilitate the tightening operation of the loop 4 around the bone tissue. To that end, the oblong element 2 may be placed in a position near to the tip of end 2a of the oblong element 2 of a similar passage to that which allows the loop 4 to be formed. Likewise, this passage and the strip of the oblong element 2 may be equipped with complementary means of retention to prevent the pulling grip 9 becoming undone when pulling on it.

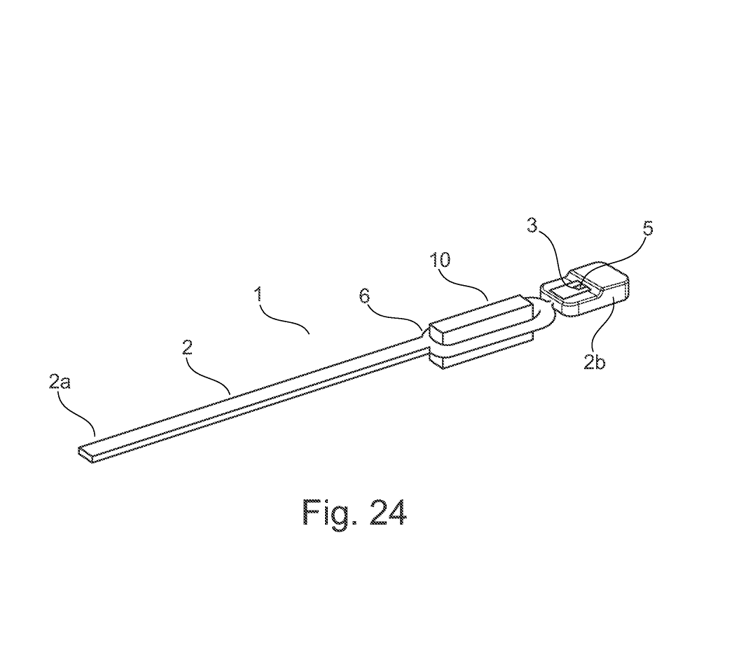

FIG. 24 shows an example of device 1 that is especially fit to take advantage of the pseudoelastic properties of the active strip 6. According to this variation, the active strip 6 is subject to effort that causes recoverable stretching of the material forming the active strip 6. Device 1 is applied to the bone tissue with a component 10 that exerts a force on the active strip 6 to keep it stretched, that component being adapted for handling in order for it to cease to act on the active strip 6 once the loop 4 is formed, which would cause the active strip 6 to recover its nominal shape, shortening the perimeter of the loop 4. The active strip 6 may be formed by titanium or nitinol.

While the above description contains many specifics, those specifics should not be construed as limitations on the scope of the disclosure, but merely as exemplifications of preferred implementations thereof. Moreover, for the sake of clarity, not every conceivable combination has been disclosed. However, it is appreciated that many of the features disclosed herein are interchangeable among the various implementations. Those skilled in the art will envision many other possible variations that are within the scope and spirit of the disclosure.

What is claimed is:

1. An apparatus for tightly securing together first and second bone segments having peripheral surfaces comprising one or more bends, the apparatus comprising:
an elongate flexible strap having a proximal end and a distal end and capable of being formed and maintained in a loop configuration about the first and second bone segments; and
the elongate flexible strap having a first elongate portion situated between the proximal end and the distal end, the first elongate portion of the elongate flexible strap being more flexible than one or more other portions of the elongate flexible strap, the first elongate portion configured to extend across at least one of the one or more bends of one or both of the first and second bone segments when the apparatus is tightly looped about the first and second bone segments.

2. An apparatus according to claim 1, wherein when the apparatus is in a rest state the first elongate portion includes a curve, the shape of the curve configured to cause at least a portion of the first elongate portion to extend toward a peripheral face of one or both of the first and second bone segments when the elongate flexible strap is initially looped about the first and second bone segments, the curve configured to cause the elongate flexible strap to form a loop about the first and second bone segments that when tightened has a perimeter that is smaller than is otherwise achievable without the existence of the curve with respect to the perimeter of the cross-section of the joined first and second bone segments.

3. An apparatus according to claim 1, wherein the strap includes a top face and a bottom face, the first elongate portion comprising one or more through openings extending between the top and bottom faces, the one or more through openings causing the first elongate portion of the strap to be more flexible than the one or more other portions of the elongate flexible strap.

4. An apparatus according to claim 1, wherein the strap includes a top face and a bottom face, the first elongate portion comprising an elongate through opening extending between the top and bottom faces, the elongate through opening causing the first elongate portion of the elongate flexible strap to be more flexible than the one or more other portions of the elongate flexible strap.

5. An apparatus according to claim 4, wherein the elongate through opening has a trapezoidal cross-section with the opening being wider at the top face of the elongate flexible strap than at the bottom face of the elongate flexible strap.

6. An apparatus according to claim 1, wherein the elongate flexible strap includes a top face and a bottom face, the first elongate portion comprising one or more openings extending partially between the top and bottom faces, the one or more openings causing the first elongate portion of the elongate flexible strap to be more flexible than the one or more other portions of the elongate flexible strap.

7. An apparatus according to claim 6, wherein the one or more openings are disposed in the top face.

8. An apparatus according to claim 1, wherein the overall width of the elongate flexible strap along the first elongate portion is greater than the overall width of the one or more other portions of the elongate flexible strap.

9. An apparatus for tightly securing together first and second bone segments having peripheral surfaces comprising one or more bends, the apparatus comprising:
an elongate flexible strap having a proximal end and a distal end, the elongate flexible strap having a plurality of ratchet teeth disposed along an elongate distal portion thereof;
a locking head situated at the proximal end of the elongate flexible strap, the head defining a transverse channel with a ratchet member situated therein, the channel dimensioned to receive the elongate distal portion of the elongate flexible strap, the ratchet member having one or more teeth adapted to engage the ratchet teeth of the elongate flexible strap in a manner that permits movement of the elongate distal portion of the elongate flexible strap through the channel in only one direction, the elongate distal portion of the elongate flexible strap adapted to be advanced through the channel until the apparatus assumes a secured state with the first and second bone segments being tightly secured together;
the elongate flexible strap having a first elongate portion situated between the locking head and the elongate distal portion, the first elongate portion of the elongate flexible strap being more flexible than the elongate distal portion of the elongate flexible strap, the first elongate portion configured to extend across at least one of the one or more bends of the first and/or second bone segment when the apparatus is in the secured state.

10. An apparatus according to claim 9, wherein when the apparatus is in a rest state the first elongate portion includes a curve, the shape of the curve configured to cause at least a portion of the first elongate portion to extend toward a peripheral face of one or both of the first and second bone segments when the elongate flexible strap is initially loosely looped about the first and second bone segments, the curve configured to cause the elongate flexible strap to form a loop about the first and second bone segments that when tightened has a perimeter that is smaller than is otherwise achievable without the existence of the curve with respect to the perimeter of the cross-section of the joined first and second bone segments.

11. An apparatus according to claim 9, wherein the elongate flexible strap includes a top face and a bottom face, the plurality of ratchet teeth being situated along the top face.

12. An apparatus according to claim 9, wherein the channel extends between top and bottom faces of the locking head and has an inlet at the bottom face of the locking head and an outlet at the top face of the locking head.

13. An apparatus according to claim 9, wherein the strap includes a top face and a bottom face, the first elongate portion comprising one or more through openings extending between the top and bottom faces, the one or more through openings causing the first elongate portion of the elongate flexible strap to be more flexible than the elongate distal portion of the elongate flexible strap.

14. An apparatus according to claim 9, wherein the strap includes a top face and a bottom face, the first elongate portion comprising an elongate through opening extending between the top and bottom faces, the elongate through opening causing the first elongate portion of the elongate flexible strap to be more flexible than the elongate distal portion of the elongate flexible strap.

15. An apparatus according to claim 14, wherein the elongate through opening has a trapezoidal cross-section with the opening being wider at the top face of the elongate flexible strap than at the bottom face of the elongate flexible strap.

16. An apparatus according to claim 9, wherein the elongate flexible strap includes a top face and a bottom face, the first elongate portion comprising one or more openings extending partially between the top and bottom faces, the one or more openings causing the first elongate portion of the elongate flexible strap to be more flexible than the elongate distal portion of the elongate flexible strap.

17. An apparatus according to claim 16, wherein the one or more openings are disposed in the top face.

18. An apparatus according to claim 9, wherein the overall width of the elongate flexible strap along the first elongate portion is greater than the overall width of the elongate distal portion.

19. An apparatus according to claim 9, wherein the elongate distal portion of the elongate flexible strap comprise features that facilitate the formation of a loop therein suitable for receiving and being gripped by one or more fingers of a human hand.

20. An apparatus for tightly securing together a first bone segment with a second bone segment, the peripheral surface of each of the first and second bone segments including a top face, a bottom face and a side face interposed between the respective top and bottom faces, the side face of the second bone segment being located opposite the side face of the first bone segment, a first corner existing between the top and side faces of the first bone segment, a second corner existing between the top and side faces of the second bone segment, a third corner existing between the bottom and side faces of the second bone segment, a fourth corner existing between the bottom and side faces of the first bone segment, the apparatus comprising:
an elongate flexible strap having a proximal end and a distal end, the elongate flexible strap having a plurality of ratchet teeth disposed along an elongate distal portion thereof;
a locking head situated at the proximal end and unitarily formed as a single piece with the elongate flexible strap, the head defining a transverse channel with a ratchet member situated therein, the channel dimensioned to receive the elongate distal portion of the elongate flexible strap, the ratchet member having one or more teeth adapted to engage the ratchet teeth of the elongate flexible strap in a manner that permits movement of the elongate distal portion of the elongate flexible strap through the channel in only one direction, the elongate distal portion of the elongate flexible strap adapted to be advanced through the channel until the apparatus assumes a secured state with the first and second bone segments being tightly secured together, the locking head being configured to rest on or near the first corner of the first bone segment when the apparatus is in the secured state;
the elongate flexible strap having a first elongate portion situated between the locking head and the elongate distal portion, the first elongate portion of the elongate flexible strap being more flexible than the elongate distal portion of the elongate flexible strap, the first elongate portion configured to extend across at least the second and third corners of the second bone segment when the apparatus is in the secured state.

21. An apparatus according to claim 20, wherein the first elongate portion is configured to extend across at least the second and third corners of the second bone segment and also across at least a portion of the bottom face of the second segment when the apparatus is in the secured state.

22. An apparatus according to claim 20, wherein the first elongate portion is configured to extend across at least the second and third corners of the second bone segment and also across the bottom face of the second segment and at least a portion of the bottom face of the first segment when the apparatus is in the secured state.

23. An apparatus according to claim 20, wherein when the apparatus is in a rest state the first elongate portion includes a curve, the shape of the curve configured to cause at least a portion of the first elongate portion to extend toward a peripheral face of one or both of the first and second bone segments when the elongate flexible strap is initially loosely looped about the first and second bone segments, the curve configured to cause the elongate flexible strap to form a loop about the first and second bone segments that when tightened has a perimeter that is smaller than is otherwise achievable without the existence of the curve with respect to the perimeter of the cross-section of the joined first and second bone segments.

24. An apparatus according to claim 20, wherein the elongate flexible strap includes a top face and a bottom face, the plurality of ratchet teeth being situated along the top face.

25. An apparatus according to claim 20, wherein the channel extends between top and bottom faces of the locking head and has an inlet at the bottom face of the locking head and an outlet at the top face of the locking head.

26. An apparatus according to claim 20, wherein the strap includes a top face and a bottom face, the first elongate portion comprising one or more through openings extending between the top and bottom faces, the one or more through openings causing the first elongate portion of the strap to be more flexible than the elongate distal portion of the elongate flexible strap.

27. An apparatus according to claim 26, wherein the strap includes a top face and a bottom face, the first elongate portion comprising an elongate through opening extending between the top and bottom faces, the elongate through opening causing the first elongate portion of the elongate flexible strap to be more flexible than the elongate distal portion of the elongate flexible strap.

28. An apparatus according to claim 20, wherein the elongate through opening has a trapezoidal cross-section with the opening being wider at the top face of the elongate flexible strap than at the bottom face of the elongate flexible strap.

29. An apparatus according to claim 20, wherein the elongate flexible strap includes a top face and a bottom face, the first elongate portion comprising one or more openings extending partially between the top and bottom faces, the one or more openings causing the first elongate portion of the elongate flexible strap to be more flexible than the elongate distal portion of the elongate flexible strap.

30. An apparatus according to claim 29, wherein the one or more openings are disposed in the top face.

31. An apparatus according to claim 20, wherein the overall width of the elongate flexible strap along the first elongate portion is greater than the overall width of the elongate distal portion.

32. An apparatus according to claim 20, wherein the elongate distal portion of the elongate flexible strap comprise features that facilitate the formation of a loop therein suitable for receiving and being gripped by one or more fingers of a human hand.
